(19) United States
(12) Patent Application Publication
(10) Pub. No.: US 2025/0387041 A1
(43) Pub. Date: Dec. 25, 2025

(54) REJECTING SPURIOUS MAGNETIC FIELDS DURING MAGNETIC MARKER LOCALIZATION

(71) Applicant: Stryker Corporation, Portage, MI (US)

(72) Inventors: Prashant PANDEY, Toronto (CA); John DILLON, Mississauga (CA); Ananth RAVI, Toronto (CA); Mark SEMPLE, Toronto (CA)

(73) Assignee: Stryker Corporation, Portage, MI (US)

(21) Appl. No.: 19/236,768

(22) Filed: Jun. 12, 2025

Related U.S. Application Data
(60) Provisional application No. 63/662,397, filed on Jun. 20, 2024.

(51) Int. Cl. A61B 5/06; A61B 5/00; A61B 90/00

(52) U.S. Cl. CPC A61B 5/062; A61B 5/7203; A61B 5/7264; A61B 5/742; A61B 2090/3954

(57) ABSTRACT

An exemplary method for detecting magnetic noise comprises: receiving a set of magnetic field gradient values obtained using a probe device; providing the set of magnetic field gradient values to a neural network to predict a marker state of a magnetic marker; providing the predicted marker state from the neural network to a magnetic field gradient prediction model to generate a predicted set of magnetic field gradient values; comparing the set of magnetic field gradient values obtained using the probe device with the set of predicted magnetic field gradient values; and providing a magnetic noise indicator depending on the comparison between the set of magnetic field gradient values obtained using the probe device and the set of predicted magnetic field gradient values.

### CROSS-REFERENCE TO RELATED APPLICATION

This application claims the benefit of U.S. Provisional Application 63/662,397 filed on Jun. 20, 2024, the entire content of which is incorporated herein by reference for all purposes.

### FIELD OF THE DISCLOSURE

The present disclosure relates to surgical localization, and more particularly, to the techniques for localization of magnetic markers.

### BACKGROUND OF THE DISCLOSURE

Localizing a magnetic marker using a probe device (e.g., a wand) having one or more magnetic sensors can lead to inaccurate measurements in the presence of metal instruments (such as surgical retractors and clamps), which may themselves exhibit a significant magnetic field and/or affect nearby magnetic fields. There is a need for the ability to detect and reject such spurious signals to accurately localize the magnetic marker-sometimes called “noise rejection” or “noise cancellation” in this disclosure.

### BRIEF SUMMARY

An exemplary method for detecting magnetic noise by a magnetic marker localization system comprises: receiving a set of magnetic field gradient values obtained using a probe device of the magnetic marker localization system; providing the set of magnetic field gradient values to a neural network, wherein the neural network is trained to receive the set of magnetic field gradient values and predict a marker state of a magnetic marker; providing the predicted marker state from the neural network to a magnetic field gradient prediction model, wherein the magnetic field gradient prediction model is configured to receive the predicted marker state and generate a predicted set of magnetic field gradient values; comparing the set of magnetic field gradient values obtained using the probe device with the set of predicted magnetic field gradient values; and providing a magnetic noise indicator depending on the comparison between the set of magnetic field gradient values obtained using the probe device and the set of predicted magnetic field gradient values.

In some examples, comparing the set of magnetic field gradient values obtained using the probe device with the set of predicted magnetic field gradient values comprises calculating one or more similarity measures. The one or more similarity measures can comprise a normalized error vector magnitude, a cosine similarity, or a combination thereof. The magnetic noise indicator can be provided if the normalized error vector is greater than a first predetermined threshold or when the cosine similarity is greater than a second predetermined threshold. The one or more similarity measures cab comprise one or more magnitude differences, one or more angular differences, or any combination thereof.

In some examples, the method further comprises providing one or more historical pose measurements of the probe device to the neural network. The one or more historical pose measurements can comprise one or more position measurements, one or more orientation measurements, or a combination thereof.

In some examples, the magnetic field gradient prediction model comprises a neural network model. In some examples, the magnetic field gradient prediction model comprises an equation. In some examples, the magnetic noise indicator can comprise a visual indicator, an audible indicator, and/or a tactile indicator. The method can further comprise: changing a color or a shape for the visual indicator based on the comparison.

In some examples, the neural network or the magnetic field gradient prediction model is selected based on a probe device type associated with the probe device. In some examples, the neural network or the magnetic field gradient prediction model is selected based on a magnetic marker type associated with the magnetic marker. In some examples, the comparison between the set of magnetic field gradient values obtained using the probe device and the set of predicted magnetic field gradient values is based on one or more predetermined criteria. The method can further comprise calculating a distance between the magnetic marker and the probe device; and updating the predetermined one or more criteria for detecting magnetic noise for the next iteration.

An exemplary non-transitory computer-readable storage medium can store one or more programs for detecting magnetic noise by a magnetic marker localization system, the one or more programs comprising instructions, which when executed by one or more processors of an electronic device, cause the electronic device to perform any of the methods described herein or any combination of the methods.

An exemplary magnetic marker localization system comprises: a probe device having one or more magnetic sensors; and a processor in electronic communication with the probe device, the processor programmed to: perform any of the methods described herein or any combination of the methods.

### DESCRIPTION OF THE DRAWINGS

For a fuller understanding of the nature and objects of the disclosure, reference should be made to the following detailed description taken in conjunction with the accompanying drawings.

FIG. 1 is a diagram showing the inputs and outputs of two neural networks used in noise rejection according to an embodiment of the present disclosure.

FIG. 2 is a diagram showing how similarity is measured between any pair of magnetic field gradient measurements and predictions according to an embodiment of the present disclosure.

FIG. 3 illustrates an exemplary process for detecting magnetic noise by a magnetic marker localization system, in accordance with some examples.

FIG. 4A illustrates an exemplary magnetic marker localization system, in accordance with some examples.

FIG. 4B illustrates use of an exemplary magnetic marker localization system, in accordance with some examples.

FIG. 5A illustrates exemplary operations of a magnetic marker localization system, in accordance with some examples.

FIG. 5B illustrates exemplary operations of a magnetic marker localization system, in accordance with some examples.

FIG. 6 illustrates an exemplary electronic device, in accordance with some examples.

### DETAILED DESCRIPTION OF THE DISCLOSURE

Disclosed herein are systems, electronic devices, methods, non-transitory storage media, and apparatuses for detecting magnetic noise by a magnetic marker localization system. An exemplary magnetic marker localization system may detect magnetic noise generated by objects other than a magnetic marker in the surgical environment, such as surgical tools, which may cause erroneous measurements by the magnetic marker localization system. An exemplary magnetic marker localization system can receive a set of magnetic field gradient values from a probe device of the magnetic marker localization system. The magnetic marker localization system can provide the set of magnetic field gradient values to a neural network. The neural network is trained to receive the set of magnetic field gradient values and predict a marker state of a magnetic marker. The predicted marker state from the neural network is provided to a magnetic field gradient prediction model, which is configured to receive the predicted marker state and generate a predicted set of magnetic field gradient values. The magnetic marker localization system can compare the set of magnetic field gradient values obtained using the probe device with the set of predicted magnetic field gradient values and provide a magnetic noise indicator based on the comparison. By leveraging trained machine-learning models such as neural network models, examples of the present disclosure are configured to be lightweight and efficient such that frequent iterations of the magnetic noise detection can be performed to provide real-time or near-real-time detection of magnetic noise in the surgical environment.

Localizing a magnetic marker using a probe device (e.g., a wand) having one or more magnetic sensors can lead to inaccurate measurements in the presence of metal instruments (such as surgical retractors, clamps, etc.), which themselves may exhibit a significant magnetic field (e.g., due to the metallic material, due to use and/or degradation). During localization, it is possible for a magnetic marker localization system to output a numerical distance value, a target position, and provide auditory feedback when the probe device is near a surgical instrument. This can cause significant confusion to the surgeon, who may interpret this output as the probe device tracking the magnetic marker instead, and therefore perform an inaccurate procedure. This problem may be exacerbated when a tip of the probe device and surgical instruments are in a surgically created cavity where it is not possible to visually confirm whether the probe device tip is in proximity to any metal instruments. As a result, surgeons may have to move the probe device around or remove some/all instruments or replace existing instruments with new instruments that are not magnetized in an attempt to resolve the problem, which can lead to disruptions to surgical workflows and protocols and compromise patient safety. Because it may be difficult to differentiate whether the localization system is localizing a marker or an instrument, clinical situations may arise where it becomes difficult to trust the output of the system.

The present disclosure provides techniques for noise rejection during localization of a magnetic marker. Through noise rejection, a localization system can include a mode wherein, for example, the system communicates to the surgeon that they are in proximity to a metal instrument or that a location of the marker cannot be reliably determined due to noise. As a result, the surgeon can more efficiently resolve the issue by moving any instruments away from the probe device tip.

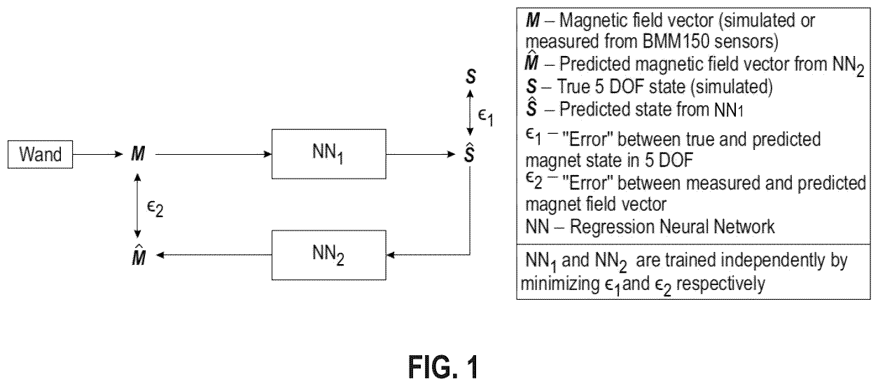

In an embodiment, two neural networks (NN) may be trained using a training set of data (e.g., simulated data, etc.) The first neural network (NN₁) is trained to predict a state of a magnetic marker (Ŝ) given magnetic field gradient measurements (M). The state of the marker may be predicted in five degrees-of-freedom (3 position states and 2 orientation states). The second neural network (NN₂) is trained to solve the inverse problem—i.e., predicting magnetic field gradient measurements (\hat{M}) given a state of the magnetic marker (S), which includes all five degrees-of-freedom. The networks are independently trained by minimizing the R² loss between the true and predicted states and measurements respectively, denoted as ϵ₁ and ϵ₂ in FIG. 1. The first and second neural networks may be trained for a known probe device configuration (e.g., positions, orientations, characteristics, etc. of one or more magnetic sensors) and for a known marker configuration (e.g., size, shape, material, magnetic characteristics, etc.)

In use, a magnetic marker may be localized using the first neural network NN₁ on its own. When noise rejection is desired, the system may be configured to use the predicted magnetic marker state from NN₁ as an input to NN₂. In this way, NN₂ will provide (output) predicted magnetic field gradient values for the predicted magnetic marker state from NN₁. The measured magnetic field gradient values (M) and the predicted magnetic field gradient values (\hat{M}) are compared.

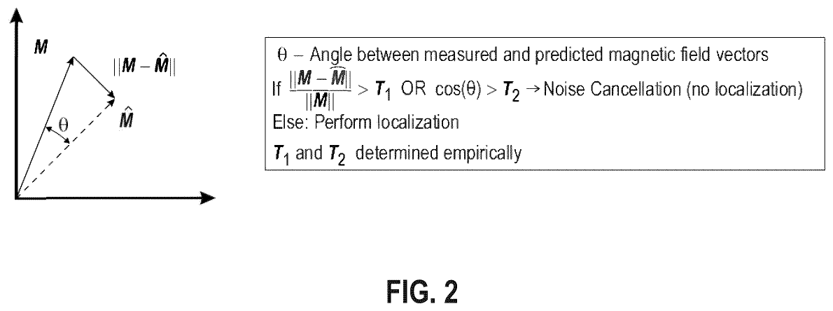

For example, the measured magnetic field gradient values (M) and the predicted magnetic field gradient values (\hat{M}) may be compared using two similarity measurements (see FIG. 2) to establish how similar the predicted values are to the measured values for the given predicted state (Ŝ). The first similarity measure is the normalized error vector magnitude, defined as:

$$\frac{‖ M - \hat{M} ‖}{‖ M ‖} ,$$

which represents the magnitude of the error vector M−\hat{M} as a proportion of the magnitude of the measured magnetic field M. The second similarity measure may be a cosine similarity, which measures the cosine of the angle between the two vectors. The cosine similarity may be advantageous due to its performance in being able to measure similarities between high-dimensional vector spaces (the magnetic field gradient vectors may be, for example, 15-dimensional). When the two similarity measures meet threshold criteria (e.g., predetermined criteria), the localization system will “reject” the measurements and prevent any localization. For example, in this “rejected” state, a user interface may display a gray circle with no distance readings or other messages. Other techniques (visual, audible, tactile, etc.) and combinations of techniques may be used to indicate the ‘rejected’ state.

In some embodiments, it may be easier to detect and reject magnetic field measurements produced by instruments that have a significantly different characteristic to the magnetic marker, due to the definition of the similarity measures. For example, large and strong permanent magnets, or very large metal instruments (such as DeBakey forceps), produce a field that is easily distinguishable from a typical magnetic marker and therefore can be rejected as far as 20-40 cm from the probe device tip. Conversely, smaller instruments (such as retractors) or those that have a field similar to the magnetic marker may require that the instrument be closer to the probe device tip (approximately 1-10 cm) before the noise rejection is activated. In some examples, the system can identify the surgical instruments in the surgical environment and adjust the sensitivity of the magnetic noise detection accordingly. The surgical instruments may be identified based on one or more user inputs (e.g., a user input specifying the surgical instruments being used, a user input specifying the type of surgical procedure being performed). Additionally or alternatively, the surgical instruments may be identified automatically (e.g., based on computer vision, based on electronic devices that the surgical instruments are connected to). The system may adjust any aspect of the magnetic noise detection, such as the models used (e.g., NN₁, NN₂), the calculation of similarity measures, and the thresholds (T₁, T₂). For example, the system can decrease the thresholds if the surgical instruments are smaller or more similar to the magnetic marker, so as to increase the sensitivity of the magnetic noise detection.

The two thresholds for normalized error vector magnitude (T₁) and cosine similarity (T₂) may be chosen empirically based on measurements made of a magnetic marker and with the applicable localization system in the presence of representative surgical tools (Allis clamp, DeBakey forceps, retractors, etc.) In some examples, T₁ may be 1, 2 (i.e., M is different by a vector length of 2× itself), 3 (i.e., \hat{M} is different by a vector length of 3× itself), or the like. For example, a group of users (e.g., surgeons) may be offered the use of the noise rejection software with sets of pre-selected thresholds for the two similarity metrics, and the users are asked which threshold (or range of thresholds) they prefer when localizing the marker in the presence of representative surgical tools.

In an aspect, the present disclosure may be embodied as a method for noise cancellation in a magnetic marker localization system. The method includes receiving a set of magnetic field gradient values (M) from a probe device of a localization system. A predicted marker state (Ŝ) is generated by providing the magnetic field gradient values as an input to a first neural network. The first neural network is trained to locate a magnetic marker using magnetic field gradient values of the probe device. The marker state may be determined in five degrees-of-freedom. The method includes generating a set of predicted magnetic field gradient values (\hat{M}) by providing the predicted marker state (Ŝ) from the first neural network as an input to a second neural network. The second neural network is trained to predict magnetic field gradient values using a location of a magnetic marker.

The set of magnetic field gradient values received from the probe device is compared with the set of predicted magnetic field gradient values to determine a similarity. For example, comparing the set of magnetic field gradient values received from the probe device with the set of predicted magnetic field gradient values may include calculating a normalized error vector magnitude, calculating a cosine similarity, or both.

A rejection indicator is provided when the similarity meets one or more predetermined criteria. For example, the rejection indicator is provided when the normalized error vector is greater than a first predetermined threshold or when the cosine similarity is greater than a second predetermined threshold. The rejection indicator may be a mode wherein the localization system provides no measurement. The rejection indicator may be a visual indicator, an audible indicator, and/or a tactile indicator.

In another aspect, the present disclosure may be embodied as a magnetic marker localization system. The localization system may include a probe device having one or more magnetic sensors. A processor may be in electronic communication with the probe device. The processor may be programmed to perform any method disclosed herein.

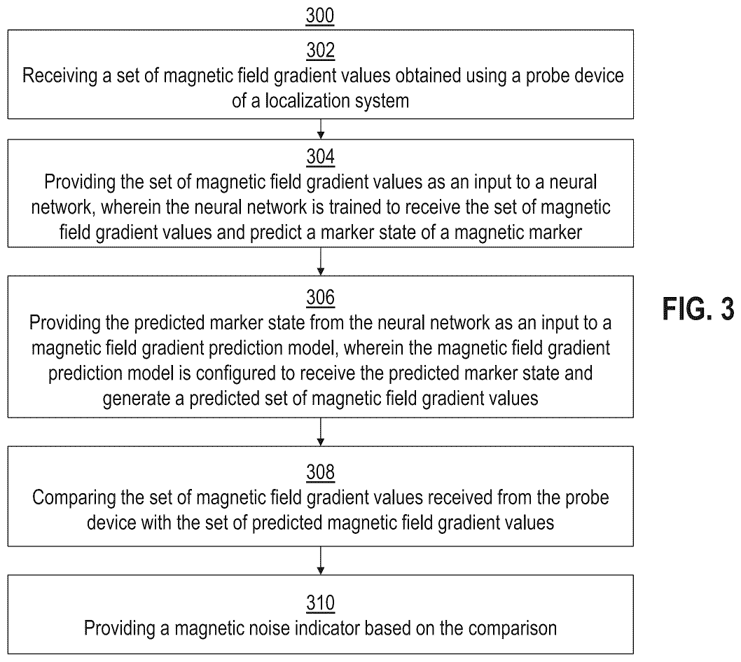

FIG. 3 illustrates an exemplary process 300 for detecting magnetic noise by a magnetic marker localization system, in accordance with some examples. Process 300 is performed, for example, using one or more electronic devices of the magnetic marker localization system, such as system 400 as described below. The process 300 can be performed over multiple iterations (e.g. periodically) to detect the presence of magnetic noise. In some examples, the process 300 can be performed each time a set of magnetic field gradient values is received (i.e., at the same measurement rate as the probe device). The models and the operations in the process 300 are configured to be lightweight and efficient such that frequent iterations of the process 300 can be performed to provide real-time or near-real-time detection of magnetic noise in the surgical environment.

In some examples, process 300 may be performed using a client-server system, and the blocks of process 300 can be divided up in any manner between the server and one or more client devices. Thus, while portions of process 300 are described herein as being performed by particular devices, it will be appreciated that process 300 is not so limited. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail below) are exemplary by nature and, as such, should not be viewed as limiting.

At block 302, an exemplary magnetic marker localization system (e.g., one or more electronic devices) receives a set of magnetic field gradient values (e.g., M in FIG. 1) obtained using a probe device (e.g., a wand) of the magnetic marker localization system. The system can receive the set of magnetic field gradient values by receiving measurements from the probe device and calculating the set of magnetic field gradient values using the measurements. In some examples, the probe device provides magnetic field measurements (e.g., indicative of how strong the magnetic field is and in which way it is pointing as measured by each sensor) to the system. The system can calculate the magnetic gradient values by subtracting the sensor measurements from each other to remove the earth's magnetic field.

At block 304, the system provides the set of magnetic field gradient values as an input to a neural network (e.g., NN₁ in FIG. 1). The neural network is trained to receive the set of magnetic field gradient values and predict a marker state of a magnetic marker (e.g., Ŝ in FIG. 1). In some examples, the system can use a different type of model to predict the marker state, such as a gradient descent optimization model. It should be appreciated that a neural network may be relatively lightweight and computationally efficient and thus can be deployed in the magnetic marker localization system to predict the marker state in an accurate and fast manner without requiring additional compute or memory resources. The predicted marker state is indicative of one or more aspects of the position of the magnetic marker. In some examples, the predicted marker state is in five degrees-of-freedom.

In some examples, the system may provide additional inputs to the neural network to improve the accuracy of the prediction of the marker state, such as pose measurements of the probe device. Pose measurements can include location measurements and/or orientation measurements that indicate the position of the probe device in space. Pose measurements may be obtained via one or more pose sensors in the probe device, such as one or more gyroscopes, one or more accelerometers, or the like. In some examples, the system can provide a series of pose measurements corresponding to a plurality of time points, such as x number of most recently acquired pose measurements by the one or more pose sensors, pose measurements (direct or interpolated) corresponding to the time points associated with the last x number of iterations, or the like. The historical pose information (and accordingly the trajectory information) of a probe device can be substantially different from that of a surgical instrument. Thus, the addition of pose information as input into the neural network can improve the accuracy of the neural network.

At block 306, the system provides the predicted marker state (e.g., Ŝ in FIG. 1) from the neural network as an input to a magnetic field gradient prediction model (e.g., NN₂ in FIG. 1). The magnetic field gradient prediction model is configured to receive the predicted marker state and generate a predicted set of magnetic field gradient values (e.g., \hat{M} in FIG. 1). In some examples, the magnetic field gradient prediction model is a neural network model such as NN₂ in FIG. 1. In some examples, the magnetic field gradient prediction model comprises a physics equation (e.g., T₁, T₂) such as the exemplary equation below.

$$B ( r ) = ∇ \times A = \frac{{μ}_{0}}{4 π} [ \frac{3 r ( m \cdot r )}{{r}^{5}} - \frac{m}{{r}^{3}} ]$$

In some examples, the system may provide the magnetic noise indicator if any one of the similarity measures meets a corresponding threshold. For example, the system may provide the magnetic noise indicator if the normalized error vector is greater than a first predetermined threshold or when the cosine similarity is greater than a second predetermined threshold.

In other examples, the system may provide the magnetic noise indicator if multiple similarity measures all meet their respective thresholds. In other examples, the system may provide the magnetic noise indicator if a combination (e.g., linear combination, non-linear combination) of some or all of the similarity measures meets a corresponding threshold.

In some examples, providing the magnetic noise indicator can comprise making one or more objects appear, changing the appearance of the one or more objects, making the one or more objects disappear, or any combination thereof.

The magnetic noise indicator may comprise a visual indicator, an audible indicator, and/or a tactile indicator. In some examples, the system may determine a visual characteristic (e.g., color, shape, size) for the visual indicator to indicate the amount of magnetic noise detected, which in turn can be estimated by the magnitude of the similarity. For example, the system may determine the color of the visual indicator based on the one or more similarity measures—a smaller difference (e.g., less than 1× similarity measures) may indicate a lower magnetic noise and thus is associated with a less alarming color (e.g., green, lighter red), while a larger difference (e.g., greater than 1× similarity measures) may indicate a higher magnetic noise and thus is associated with a more alarming color (e.g., red, darker red). As another example, the system may determine the shape of the visual indicator based on the one or more similarity measures—a smaller difference may indicate a lower magnetic noise and thus is associated with a less alarming shape (e.g., perfect circle), while a larger difference may indicate a higher magnetic noise and thus is associated with a more alarming shape (e.g., spikey circle). In some examples, the system may determine a recommendation (e.g., remove or move the instrument) if the one or more similarity measures meet one or more predetermined criteria and continue to provide the recommendation unless the similarity measures no longer meet the one or more predetermined criteria.

In some examples, the one or more predetermined criteria may be configurable by a user, thus allowing the user to adjust the sensitivity of the magnetic noise detection. A higher predetermined criteria may result in a less sensitive detection system, while a lower predetermined criteria may result in a more sensitive detection system. Depending on the surgeon, the procedure, the patient, and other factors, the user may prefer a more or less sensitive detection system. Thus, the system may allow the user to specify the various thresholds associated with the output. For example, one or more shapes on the user interface may change colors by interpolating between two color values, and the similarity measure at which this change begins and ends can be hardcoded or adjusted by the user. This range may be different if the type of feedback change is auditory instead. For example, the type of auditory tone may change from a sine wave to a square wave by interpolating between the two types.

In some examples, the system can adjust the one or more predetermined criteria (e.g., T₁, T₂) based on the distance between the magnetic marker and the probe device (e.g., based on Ŝ). When the probe device is within a predefined range of the magnetic marker, the magnetic strength from the magnetic marker becomes more dominant relative to magnetic noises (e.g., from surgical instruments). Thus, within the predefined range, it is desirable to reduce the sensitivity of the magnetic noise detection so as to not distract the user. When the probe device moves farther away from the magnetic marker, the magnetic strength from the magnetic marker becomes less dominant relative to magnetic noises, and it is desirable to increase the sensitivity of the magnetic noise detection such that magnetic inferences may be reported to the user. Accordingly, the system may update the predetermined criteria for detecting magnetic noise based on the distance between the magnetic marker and the probe device. For example, the system can increase the one or more predetermined criteria (e.g., T₁, T₂) as the distance decreases.

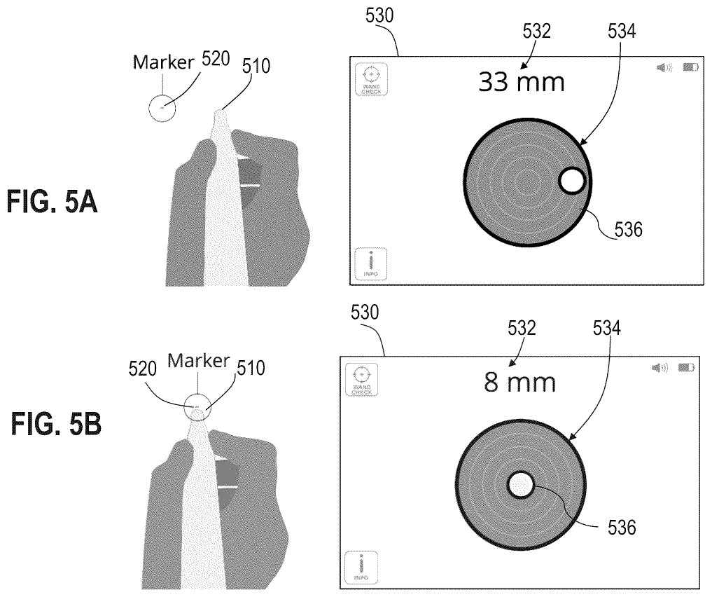

In some examples, when the probe device is determined to be within a predefined range of the magnetic marker, the system enters a target lock mode. In the target lock mode, the position of a visual indicator (e.g. 536 in FIGS. 5A-B) is locked to the bullseye target of the graphical user interface. The target lock mode is provided because if the probe device is close enough to the magnetic marker, additional movements of the visual indicator would not provide additional benefit to the user and may in fact be distracting or disruptive. In the target lock mode, the process 300 may be deactivated so as to not provide distracting or disruptive signals to the user. In some examples, the process 300 is still activated but the predetermined criteria for detecting magnetic noise are increased. For example, the system can increase the one or more predetermined criteria (e.g., T₁, T₂) such that a larger difference in the one or more similarity measurements (e.g., M−\hat{M}), which indicates a higher magnetic noise, is needed for the system to determine that a magnetic noise is detected and output an alert.

The system exits the target lock mode if the system determines that that probe device is outside of the predefined range of the magnetic marker. When the system is not in the target lock mode, the visual indicator is unlocked and its coordinates may change to indicate the magnetic marker's relative position to the probe device tip. Further, the system can decrease the one or more predetermined criteria (e.g., T₁, T₂) to increase to the sensitivity of the magnetic noise detection.

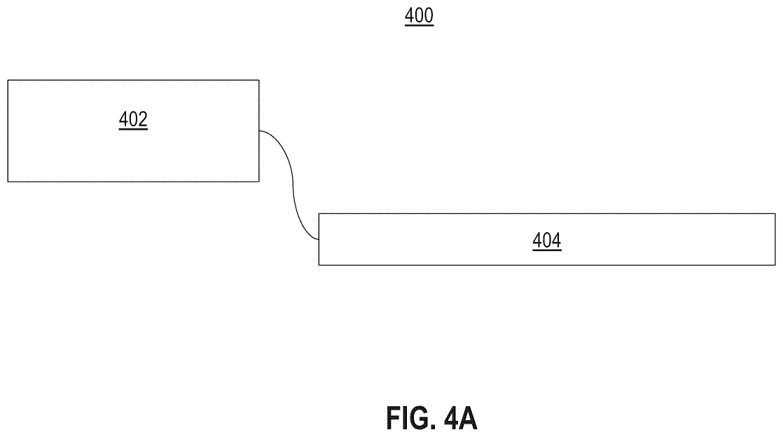

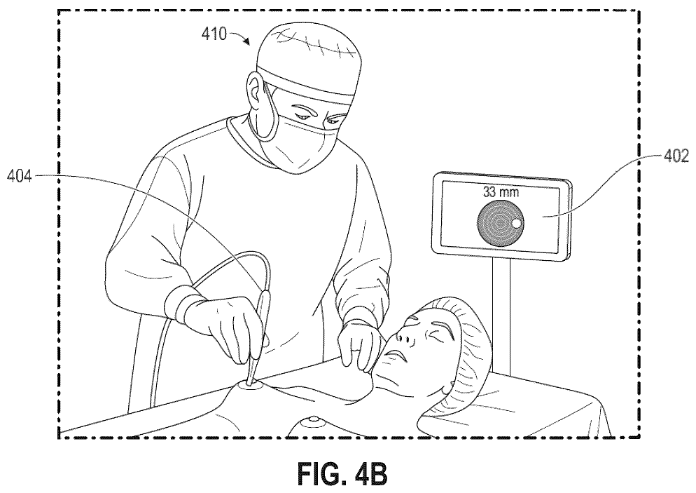

FIG. 4A illustrates an exemplary magnetic marker localization system, in accordance with some embodiments. The exemplary magnetic marker localization system 400 comprises an electronic device 402 and a probe device 404 comprising one or more magnetic sensors. The electronic device 402 and the probe device 404 are communicatively coupled (e.g., via cable(s) or wirelessly) such that the probe device 404 can transmit magnetic measurements acquired by the one or more magnetic sensors to the electronic device 402 for processing. In the depicted example, the electronic device 402 is located outside of a housing of the probe device 404. In other examples, the electronic device 402 may be located within the housing of the probe device 404. The electronic device 402 can comprise one or more processors for performing some or all of the steps of the process 300. The electronic device 402 may include or may be communicatively coupled to a display for displaying outputs associated with the process 300, as described below.

FIG. 4B depicts use of an exemplary magnetic marker localization system, in accordance with some embodiments. During a surgical procedure, a surgeon 410 can maneuver the probe device 404 to detect the location of a magnetic marker implanted within a patient. The probe device 404 comprises one or more magnetic sensors that generate magnetic field gradient values, which can be provided to the electronic device 402 for determining the location of the magnetic marker. In the depicted example, the electronic device 402 is a tablet computer with a display, which can display the estimated location of the magnetic marker on a graphical user interface. In addition, the electronic device 402 can perform some or all of the steps of the process 300 to detect magnetic noise, which may originate from various surgical instruments in the surgical environment. The outputs associated with the process 300 can be provided on the display of the electronic device 402.

FIGS. 5A and 5B depict a probe tip 510 position (left) relative to a magnetic marker 520, and a corresponding result in a user interface 530 (right). In FIG. 5A, the probe tip 510 is to the right of the marker. The corresponding screen of the user interface 530 has a first indicator 532 showing a distance from the probe tip to the marker (33 mm) and a second indicator 534 showing the probe tip as a circle indicator to the right of a target center (“bullseye” target) thereby indicating the relative position. In FIG. 5B, the probe tip 510 is oriented directly above the marker 520 (i.e., the marker is in front of the probe tip). In the corresponding screen of the user interface 530, the first indicator 532 shows the distance from the probe tip to the marker (8 mm) and the second indicator 534 shows the probe tip circle at the center of the target.

In some examples, the system may determine a visual characteristic (e.g., color, shape, size) for the visual indicator 536 to indicate the amount of magnetic noise detected, which in turn can be estimated by the magnitude of the similarity. For example, the system may determine the color of the visual indicator 536 based on the one or more similarity measures—a smaller difference may indicate a lower magnetic noise and thus is associated with a less alarming color (e.g., green, lighter red), while a larger difference may indicate a higher magnetic noise and thus is associated with a more alarming color (e.g., red, darker red). As another example, the system may determine the shape of the visual indicator 536 based on the one or more similarity measures—a smaller difference may indicate a lower magnetic noise and thus is associated with a less alarming shape (e.g., perfect circle), while a larger difference may indicate a higher magnetic noise and thus is associated with a more alarming shape (e.g., spikey circle).

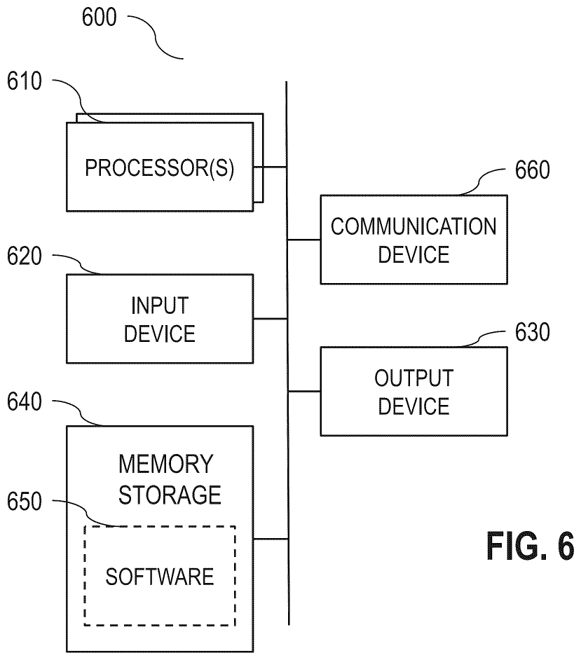

The operations described above with reference to FIG. 3 are optionally implemented by components depicted in FIG. 6. It would be clear to a person having ordinary skill in the art how other processes are implemented based on the components depicted in FIGS. 4A-6.

FIG. 6 illustrates an example of a computing device in accordance with one embodiment. Device 600 can be a host computer connected to a network. Device 600 can be a client computer or a server. As shown in FIG. 6, device 600 can be any suitable type of microprocessor-based device, such as a personal computer, workstation, server or handheld computing device (portable electronic device) such as a phone or tablet. The device can include, for example, one or more of processor 610, input device 620, output device 630, storage 640, and communication device 660. Input device 620 and output device 630 can generally correspond to those described above, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker.

Storage 640 can be any suitable device that provides storage, such as an electrical, magnetic or optical memory including a RAM, cache, hard drive, or removable storage disk. Communication device 660 can include any suitable device capable of transmitting and receiving signals over a network, such as a network interface chip or device. The components of the computer can be connected in any suitable manner, such as via a physical bus or wirelessly.

Software 650, which can be stored in storage 640 and executed by processor 610, can include, for example, the programming that embodies the functionality of the present disclosure (e.g., as embodied in the devices as described above).

Software 650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, suchr as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 600 can implement any operating system suitable for operating on the network. Software 650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the present disclosure has been described with respect to one or more particular embodiments, it will be understood that other embodiments of the present disclosure may be made without departing from the spirit and scope of the present disclosure. The following are non-limiting sample claims intended solely to illustrate some embodiments of the present disclosure.

What is claimed is:

1. A method for detecting magnetic noise by a magnetic marker localization system, the method comprising:
receiving a set of magnetic field gradient values obtained using a probe device of the magnetic marker localization system;
providing the set of magnetic field gradient values to a neural network, wherein the neural network is trained to receive the set of magnetic field gradient values and predict a marker state of a magnetic marker;
providing the predicted marker state from the neural network to a magnetic field gradient prediction model, wherein the magnetic field gradient prediction model is configured to receive the predicted marker state and generate a predicted set of magnetic field gradient values;
comparing the set of magnetic field gradient values obtained using the probe device with the set of predicted magnetic field gradient values; and
providing a magnetic noise indicator depending on the comparison between the set of magnetic field gradient values obtained using the probe device and the set of predicted magnetic field gradient values.

2. The method of claim 1, wherein comparing the set of magnetic field gradient values obtained using the probe device with the set of predicted magnetic field gradient values comprises calculating one or more similarity measures.

3. The method of claim 2, wherein the one or more similarity measures comprise a normalized error vector magnitude, a cosine similarity, or a combination thereof.

4. The method of claim 3, wherein the magnetic noise indicator is provided if the normalized error vector is greater than a first predetermined threshold or when the cosine similarity is greater than a second predetermined threshold.

5. The method of claim 2, wherein the one or more similarity measures comprise one or more magnitude differences, one or more angular differences, or any combination thereof.

6. The method of claim 1, further comprising providing one or more historical pose measurements of the probe device to the neural network.

7. The method of claim 6, wherein the one or more historical pose measurements comprise one or more position measurements, one or more orientation measurements, or a combination thereof.

8. The method of claim 1, wherein the magnetic field gradient prediction model comprises a neural network model.

9. The method of claim 1, wherein the magnetic field gradient prediction model comprises an equation.

10. The method of claim 1, wherein the magnetic noise indicator comprises a visual indicator, an audible indicator, and/or a tactile indicator.

11. The method of claim 10, further comprising: changing a color or a shape for the visual indicator based on the comparison.

12. The method of claim 1, wherein the neural network or the magnetic field gradient prediction model is selected based on a probe device type associated with the probe device.

13. The method of claim 1, wherein the neural network or the magnetic field gradient prediction model is selected based on a magnetic marker type associated with the magnetic marker.

14. The method of claim 1, wherein the comparison between the set of magnetic field gradient values obtained using the probe device and the set of predicted magnetic field gradient values is based on one or more predetermined criteria.

15. The method of claim 14, further comprising:
calculating a distance between the magnetic marker and the probe device; and
updating the predetermined one or more criteria for detecting magnetic noise for the next iteration.

16. A non-transitory computer-readable storage medium storing one or more programs for detecting magnetic noise by a magnetic marker localization system, the one or more programs comprising instructions, which when executed by one or more processors of an electronic device, cause the electronic device to:
receive a set of magnetic field gradient values obtained using a probe device of the magnetic marker localization system;
provide the set of magnetic field gradient values to a neural network, wherein the neural network is trained to receive the set of magnetic field gradient values and predict a marker state of a magnetic marker;
provide the predicted marker state from the neural network to a magnetic field gradient prediction model, wherein the magnetic field gradient prediction model is configured to receive the predicted marker state and generate a predicted set of magnetic field gradient values;
compare the set of magnetic field gradient values obtained using the probe device with the set of predicted magnetic field gradient values; and
provide a magnetic noise indicator depending on the comparison between the set of magnetic field gradient values obtained using the probe device and the set of predicted magnetic field gradient values.

17. A magnetic marker localization system, comprising:
a probe device having one or more magnetic sensors; and
a processor in electronic communication with the probe device, the processor programmed to:
receive a set of magnetic field gradient values obtained using a probe device of the magnetic marker localization system;
provide the set of magnetic field gradient values to a neural network, wherein the neural network is trained to receive the set of magnetic field gradient values and predict a marker state of a magnetic marker;
provide the predicted marker state from the neural network to a magnetic field gradient prediction model, wherein the magnetic field gradient prediction model is configured to receive the predicted marker state and generate a predicted set of magnetic field gradient values;
compare the set of magnetic field gradient values obtained using the probe device with the set of predicted magnetic field gradient values; and
provide a magnetic noise indicator depending on the comparison between the set of magnetic field gradient values obtained using the probe device and the set of predicted magnetic field gradient values.
